SEQUENCE LISTING (24 sequences)

SEQ ID NO: 1 - protein, 110 aa, Artificial Sequence
  VLCD3 Domain of DART-A
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 2 - protein, 14 aa, Artificial Sequence
  CDR1 of VLCD3 Domain of DART-A
RSSTGAVTTSNYAN

SEQ ID NO: 3 - protein, 7 aa, Artificial Sequence
  CDR2 of VLCD3 Domain of DART-A
GTNKRAP

SEQ ID NO: 4 - protein, 9 aa, Artificial Sequence
  CDR3 of VLCD3 Domain of DART-A
ALWYSNLWV

SEQ ID NO: 5 - protein, 8 aa, Artificial Sequence
  Linker 1
GGGSGGGG

SEQ ID NO: 6 - protein, 120 aa, Artificial Sequence
  VHCD123 Domain of DART-A
EVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSS

SEQ ID NO: 7 - protein, 5 aa, Artificial Sequence
  CDR1 of VHCD123 Domain of DART-A
DYYMK

SEQ ID NO: 8 - protein, 17 aa, Artificial Sequence
  CDR2 of VHCD123 Domain of DART-A
DIIPSNGATFYNQKFKG

SEQ ID NO: 9 - protein, 7 aa, Artificial Sequence
  CDR3 of VHCD123 Domain of DART-A
SHLLRAS

SEQ ID NO: 10 - protein, 113 aa, Artificial Sequence
  VLCD123 Domain of DART-A
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 11 - protein, 17 aa, Artificial Sequence
  CDR1 of VLCD123 Domain of DART-A
KSSQSLLNSGNQKNYLT

SEQ ID NO: 12 - protein, 8 aa, Artificial Sequence
  CDR2 of VLCD123 Domain of DART-A
WASTRELS

SEQ ID NO: 13 - protein, 9 aa, Artificial Sequence
  CDR3 of VLCD123 Domain of DART-A
QNDYSYPYT

SEQ ID NO: 14 - protein, 125 aa, Artificial Sequence
  VHCD3 Domain of DART-A
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 15 - protein, 4 aa, Artificial Sequence
  CDR1 of VHCD3 Domain of DART-A
TAMM

SEQ ID NO: 16 - protein, 19 aa, Artificial Sequence
  CDR2 of VHCD3 Domain of DART-A
RIRSKYNNYATYYADSVKD

SEQ ID NO: 17 - protein, 14 aa, Artificial Sequence
  CDR3 of VHCD3 Domain of DART-A
HGNFGNSYVSWFAY

SEQ ID NO: 18 - protein, 6 aa, Artificial Sequence
  Linker 2
GGCGGG

SEQ ID NO: 19 - protein, 28 aa, Artificial Sequence
  E-Coil Heterodimer-Promoting Domain
EVAALEKEVAALEKEVAALEKEVAALEK

SEQ ID NO: 20 - protein, 28 aa, Artificial Sequence
  K-Coil Heterodimer-Promoting Domain
KVAALKEKVAALKEKVAALKEKVAALKE

SEQ ID NO: 21 - protein, 272 aa, Artificial Sequence
  First Polypeptide Chain of Sequence-Optimized CD123 x CD3 Bi-specific Diabody (DART-A)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSSGGCGGGEVAALEKEVAALEKEVAALEKEVAALEK

SEQ ID NO: 22 - DNA, 816 bp, Artificial Sequence
  Polynucelotide Encoding First Polypeptide Chain of Sequence-Optimized CD123 x CD3 Bi-specific Diabody (DART-A)
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggcggaggcgaggtgcagctggtgcagtccggggctgagctgaagaaacccggagcttccgtgaaggtgtcttgcaaagccagtggctacaccttcacagactactatatgaagtgggtcaggcaggctccaggacagggactggaatggatcggcgatatcattccttccaacggggccactttctacaatcagaagtttaaaggcagggtgactattaccgtggacaaatcaacaagcactgcttatatggagctgagctccctgcgctctgaagatacagccgtgtactattgtgctcggtcacacctgctgagagccagctggtttgcttattggggacagggcaccctggtgacagtgtcttccggaggatgtggcggtggagaagtggccgcactggagaaagaggttgctgctttggagaaggaggtcgctgcacttgaaaaggaggtcgcagccctggagaaa

SEQ ID NO: 23 - protein, 280 aa, Artificial Sequence
  Second Polypeptide Chain of Sequence-Optimized CD123 x CD3 Bi-specific Diabody (DART-A)
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGKVAALKEKVAALKEKVAALKEKVAALKE

SEQ ID NO: 24 - DNA, 840 bp, Artificial Sequence
  Polynucelotide Encoding Second Polypeptide Chain of Sequence-Optimized CD123 x CD3 Bi-specific Diabody (DART-A)
gacttcgtgatgacacagtctcctgatagtctggccgtgagtctgggggagcgggtgactatgtcttgcaagagctcccagtcactgctgaacagcggaaatcagaaaaactatctgacctggtaccagcagaagccaggccagccccctaaactgctgatctattgggcttccaccagggaatctggcgtgcccgacagattcagcggcagcggcagcggcacagattttaccctgacaatttctagtctgcaggccgaggacgtggctgtgtactattgtcagaatgattacagctatccctacactttcggccaggggaccaagctggaaattaaaggaggcggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaaggatagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggaaaagtggccgcactgaaggagaaagttgctgctttgaaagagaaggtcgccgcacttaaggaaaaggtcgcagccctgaaagag

(19) United States
(12) Patent Application Publication
(10) Pub. No.: US 2021/0061911 A1
(43) Pub. Date: Mar. 4, 2021

(54) Dosing Regiments of Bi-Specific CD123 x CD3 Diabodies in the Treatment of Hematologic Malignancies

(71) Applicant: MacroGenics, Inc., Rockville, MD (US)

(72) Inventors: Jon Marc Wigginton, Rockville, MD (US); Ralph Froman Alderson, North Potomac, MD (US); Robert Joseph Lechleider, Rockville, MD (US)

(73) Assignee: MacroGenics, Inc., Rockville, MD (US)

(21) Appl. No.: 16/644,789

(22) PCT Filed: Sep. 7, 2017
(86) PCT No.: PCT/US2017/050471

§ 371 Date: Mar. 5, 2020

(51) Int. Cl. C07K 16/28; A61P 35/02

(52) U.S. Cl. CPC C07K 16/2809; C07K 16/2866; A61K 9/0019; C07K 2317/31; C07K 2317/626; A61P 35/02

(57) ABSTRACT

The present invention is directed to a dosing regimen for administering a CD123×CD3 bi-specific monovalent diabody to patients with a hematologic malignancy such as acute myeloid leukemia (AML) or myelodysplastic syndrome (MDS). The invention particularly concerns the use of such a regimen for the sequence-optimized CD 123×CD3 bi-specific monovalent diabody “DART-A,” that is capable of simultaneous binding to CD 123 and CD3.

### REFERENCE TO SEQUENCE LISTING

This application includes one or more Sequence Listings pursuant to 37 C.F.R. 1.821 et seq., which are disclosed in computer-readable media (file name: 1301-0152P_CD123_CD3_ST25.txt, created on Jul. 26, 2017, and having a size of 16,221 bytes), which file is herein incorporated by reference in its entirety.

### FIELD OF THE INVENTION:

The present invention is directed to a dosing regimen for administering a CD123×CD3 bi-specific monovalent diabody to patients with a hematologic malignancy such as acute myeloid leukemia (AML) or myelodysplastic syndrome (MDS). The invention particularly concerns the use of such a regimen for the sequence-optimized CD123×CD3 bi-specific monovalent diabody “DART-A,” that is capable of simultaneous binding to CD123 and CD3.

### BACKGROUND OF THE INVENTION:

I. CD123

CD123 (interleukin 3 receptor alpha, IL-3Rα) is a 40 kDa molecule and is part of the interleukin 3 receptor complex (Stomski, F. C. et al. (1996) “Human Interleukin-3 (IL-3) Induces Disulfide-Linked IL-3 Receptor Alpha- And Beta-Chain Heterodimerization, Which Is Required For Receptor Activation But Not High-Affinity Binding,” Mol. Cell. Biol. 16(6):3035-3046). Interleukin 3 (IL-3) drives early differentiation of multipotent stem cells into cells of the erythroid, myeloid and lymphoid progenitors. CD123 is expressed on CD34+ committed progenitors (Taussig, D. C. et al. (2005) “Hematopoietic Stem Cells Express Multiple Myeloid Markers: Implications For The Origin And Targeted Therapy Of Acute Myeloid Leukemia,” Blood 106:4086-4092), but not by CD34+/CD38− normal hematopoietic stem cells. CD123 is expressed by basophils, mast cells, plasmacytoid dendritic cells, some expression by monocytes, macrophages and eosinophils, and low or no expression by neutrophils and megakaryocytes. Some non-hematopoietic tissues (placenta, Leydig cells of the testis, certain brain cell elements and some endothelial cells) express CD123; however, expression is mostly cytoplasmic.

CD123 is reported to be expressed by leukemic blasts and leukemia stem cells (LSC) (Jordan, C. T. et al. (2000) “The Interleukin-3 Receptor Alpha Chain Is A Unique Marker For Human Acute Myelogenous Leukemia Stem Cells,” Leukemia 14:1777-1784; Jin, W. et al. (2009) “Regulation Of Th17 Cell Differentiation And EAE Induction By MAP3K NIK,” Blood 113:6603-6610) (FIG. 1). In human normal precursor populations, CD123 is expressed by a subset of hematopoietic progenitor cells (HPC) but not by normal hematopoietic stem cells (HSC). CD123 is also expressed by plasmacytoid dendritic cells (pDC) and basophils, and, to a lesser extent, monocytes and eosinophils (Lopez, A. F. et al. (1989) “Reciprocal Inhibition Of Binding Between Interleukin 3 And Granulocyte-Macrophage Colony-Stimulating Factor To Human Eosinophils,” Proc. Natl. Acad. Sci. (U.S.A.) 86:7022-7026; Sun, Q. et al. (1996) “Monoclonal Antibody 7G3 Recognizes The N-Terminal Domain Of The Human Interleukin-3 (IL-3) Receptor Alpha Chain And Functions As A Specific IL-3 Receptor Antagonist,” Blood 87:83-92; Muñoz, L. et al. (2001) “Interleukin-3 Receptor Alpha Chain (CD123) Is Widely Expressed In Hematologic Malignancies,” Haematologica 86(12):1261-1269; Masten, B. J. et al. (2006) “Characterization Of Myeloid And Plasmacytoid Dendritic Cells In Human Lung,” J. Immunol. 177:7784-7793; Korpelainen, E. I. et al. (1995) “Interferon-Gamma Upregulates Interleukin-3 (IL-3) Receptor Expression In Human Endothelial Cells And Synergizes With IL-3 In Stimulating Major Histocompatibility Complex Class II Expression And Cytokine Production,” Blood 86:176-182).

CD123 has been reported to be overexpressed on malignant cells in a wide range of hematologic malignancies including acute myeloid leukemia (AML) and myelodysplastic syndrome (MDS) (Muñoz, L. et al. (2001) “Interleukin-3 Receptor Alpha Chain (CD123) Is Widely Expressed In Hematologic Malignancies,” Haematologica 86(12):1261-1269). Overexpression of CD123 is associated with poorer prognosis in AML (Tettamanti, M. S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401).

II. CD3

CD3 is a T cell co-receptor composed of four distinct chains (Wucherpfennig, K. W. et al. (2010) “Structural Biology Of The T-Cell Receptor: Insights Into Receptor Assembly, Ligand Recognition, And Initiation Of Signaling,” Cold Spring Harb. Perspect. Biol. 2(4):a005140; pages 1-14). In mammals, the complex contains a CD3γ chain, a CD3δ chain, and two CD3_{ε} chains. These chains associate with a molecule known as the T cell receptor (TCR) in order to generate an activation signal in T lymphocytes. In the absence of CD3, TCRs do not assemble properly and are degraded (Thomas, S. et al. (2010) “Molecular Immunology Lessons From Therapeutic T-Cell Receptor Gene Transfer,” Immunology 129(2):170-177). CD3 is found bound to the membranes of all mature T cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7):913-923; Kuhns, M. S. et al. (2006) “Deconstructing The Form And Function Of The TCR/CD3 Complex,” Immunity. 2006 F eb ; 24(2): 133-139).

III. AML and MDS

AML and MDS are thought to arise in and be perpetuated by a small population of leukemic stem cells (LSCs), which are generally dormant (i.e., not rapidly dividing cells) and therefore resist cell death (apoptosis) and conventional chemotherapeutic agents. LSCs are characterized by high levels of CD123 expression, which is not present in the corresponding normal hematopoietic stem cell population in normal human bone marrow (Jin, W. et al. (2009) “Regulation Of Th17 Cell Differentiation And EAE Induction By MAP3K NIK,” Blood 113:6603-6610; Jordan, C. T. et al. (2000) “The Interleukin-3 Receptor Alpha Chain Is A Unique Marker For Human Acute Myelogenous Leukemia Stem Cells,” Leukemia 14:1777-1784). CD123 is expressed in 45%-95% of AML, 85% of Hairy cell leukemia (HCL), and 40% of acute B lymphoblastic leukemia (B-ALL). CD123 expression is also associated with multiple other malignancies/pre-malignancies: chronic myeloid leukemia (CML) progenitor cells (including blast crisis CML); Hodgkin's Reed Sternberg (RS) cells; transformed non-Hodgkin's lymphoma (NHL); some chronic lymphocytic leukemia (CLL) (CD1 1 c+), a subset of acute T lymphoblastic leukemia (T-ALL) (16%, most immature, mostly adult), plasmacytoid dendritic cell (pDC) (DC2) malignancies and CD34+/CD38− myelodysplastic syndrome (MDS) marrow cell malignancies.

AML is a clonal disease characterized by the proliferation and accumulation of transformed myeloid progenitor cells in the bone marrow, which ultimately leads to hematopoietic failure. The incidence of AML increases with age, and older patients typically have worse treatment outcomes than do younger patients (Robak, T. et al. (2009) “Current And Emerging Therapies For Acute Myeloid Leukemia,” Clin. Ther. 2:2349-2370). Unfortunately, at present, most adults with AML die from their disease.

Treatment for AML initially focuses in the induction of remission (induction therapy). Once remission is achieved, treatment shifts to focus on securing such remission (post-remission or consolidation therapy) and, in some instances, maintenance therapy. The standard remission induction paradigm for AML is chemotherapy with an anthracycline/cytarabine combination, followed by either consolidation chemotherapy (usually with higher doses of the same drugs as were used during the induction period) or human stem cell transplantation, depending on the patient's ability to tolerate intensive treatment and the likelihood of cure with chemotherapy alone (see, e.g., Roboz, G. J. (2012) “Current Treatment Of Acute Myeloid Leukemia,” Curr. Opin. Oncol. 24:711-719).

Agents frequently used in induction therapy include cytarabine and the anthracyclines. Cytarabine, also known as AraC, kills cancer cells (and other rapidly dividing normal cells) by interfering with DNA synthesis. Side effects associated with AraC treatment include decreased resistance to infection, a result of decreased white blood cell production; bleeding, as a result of decreased platelet production; and anemia, due to a potential reduction in red blood cells. Other side effects include nausea and vomiting. Anthracyclines (e.g., daunorubicin, doxorubicin, and idarubicin) have several modes of action including inhibition of DNA and RNA synthesis, disruption of higher order structures of DNA, and production of cell damaging free oxygen radicals. The most consequential adverse effect of anthracyclines is cardiotoxicity, which considerably limits administered life-time dose and to some extent their usefulness.

Thus, unfortunately, despite substantial progress in the treatment of newly diagnosed AML, 20% to 40% of patients do not achieve remission with the standard induction chemotherapy, and 50% to 70% of patients entering a first complete remission are expected to relapse within 3 years. The optimum strategy at the time of relapse, or for patients with the resistant disease, remains uncertain. Stem cell transplantation has been established as the most effective form of anti-leukemic therapy in patients with AML in first or subsequent remission (Roboz, G. J. (2012) “Current Treatment Of Acute Myeloid Leukemia,” Curr. Opin. Oncol. 24:711-719).

IV. Bi-Specific Diabodies

The provision of non-monospecific diabodies provides a significant advantage over monospecific natural antibodies: the capacity to co-ligate and co-localize cells that express different epitopes. Bi-specific diabodies thus have wide-ranging applications including therapy and immunodiagnosis. Bi-specificity allows for great flexibility in the design and engineering of the diabody in various applications, providing enhanced avidity to multimeric antigens, the cross-linking of differing antigens, and directed targeting to specific cell types relying on the presence of both target antigens. Due to their increased valency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221). Of particular importance is the co-ligating of differing cells, for example, the cross-linking of cytotoxic T cells to tumor cells (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305).

Diabody epitope binding domains may also be directed to a surface determinant of any immune effector cell such as CD3, CD16, CD32, or CD64, which are expressed on T lymphocytes, natural killer (NK) cells or other mononuclear cells. In many studies, diabody binding to effector cell determinants, e.g., Fcγ receptors (FcγR), was also found to activate the effector cell (Holliger et al. (1996) “Specific Killing Of Lymphoma Cells By Cytotoxic T-Cells Mediated By A Bispecific Diabody,” Protein Eng. 9:299-305; Holliger et al. (1999) “Carcinoembryonic Antigen (CEA)-Specific T-cell Activation In Colon Carcinoma Induced By Anti-CD3×Anti-CEA Bispecific Diabodies And B7×Anti-CEA Bispecific Fusion Proteins,” Cancer Res. 59:2909-2916; WO 2006/113665; WO 2008/157379; WO 2010/080538; WO 2012/018687; WO 2012/162068). Normally, effector cell activation is triggered by the binding of an antigen bound antibody to an effector cell via Fc-FcγR interaction; thus, in this regard, diabody molecules may exhibit Ig-like functionality independent of whether they comprise an Fc Domain (e.g., as assayed in any effector function assay known in the art or exemplified herein (e.g., ADCC assay)). By cross-linking tumor and effector cells, the diabody not only brings the effector cell within the proximity of the tumor cells but leads to effective tumor killing (see e.g., Cao et al. (2003) “Bispecific Antibody Conjugates In Therapeutics,” Adv. Drug. Deliv. Rev. 55:171-197).

However, the above advantages come at a salient cost. The formation of such non-monospecific diabodies requires the successful assembly of two or more distinct and different polypeptides (i.e., such formation requires that the diabodies be formed through the heterodimerization of different polypeptide chain species). This fact is in contrast to mono-specific diabodies, which are formed through the homodimerization of identical polypeptide chains. Because at least two dissimilar polypeptides (i.e., two polypeptide species) must be provided in order to form a non-monospecific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to prevent covalent bonding between polypeptides of the same species (i.e., so as to prevent homodimerization) (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

However, the art has recognized that bi-specific diabodies composed of non-covalently associated polypeptides are unstable and readily dissociate into non-functional monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has succeeded in developing stable, covalently bonded heterodimeric non-monospecific diabodies (see, e.g., WO 2006/113665; WO/2008/157379; WO 2010/080538; WO 2012/018687; WO/2012/162068; Johnson, S. et al. (2010) “Effector Cell Recruitment With Novel Fv-Based Dual Affinity Re-Targeting Protein Leads To Potent Tumor Cytolysis And In Vivo B-Cell Depletion,” J. Molec. Biol. 399(3):436-449; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7): 1933-1943 ; Moore, P. A. et al. (2011) “Application Of Dual Affinity Retargeting Molecules To Achieve Optimal Redirected T-Cell Killing Of B-Cell Lymphoma,” Blood 117(17):4542-4551). Such approaches involve engineering one or more cysteine residues into each of the employed polypeptide species. For example, the addition of a cysteine residue to the C-terminus of such constructs has been shown to allow disulfide bonding between the polypeptide chains, stabilizing the resulting heterodimer without interfering with the binding characteristics of the bivalent molecule.

Notwithstanding such success, the production of stable, functional heterodimeric, non-monospecific diabodies can be further optimized by the careful consideration and placement of cysteine residues in one or more of the employed polypeptide chains. Such optimized diabodies can be produced in higher yield and with greater activity than non-optimized diabodies. The present invention is thus directed to the problem of providing polypeptides that are particularly designed and optimized to form heterodimeric diabodies. The invention solves this problem through the provision of exemplary, optimized CD123×CD3 diabodies.

### SUMMARY OF THE INVENTION

The present invention is directed to a dosing regimen for administering a CD123×CD3 bi-specific monovalent diabody to patients with a hematologic malignancy such as acute myeloid leukemia (AML) or myelodysplastic syndrome (MDS). The invention particularly concerns the use of such a regimen for the sequence-optimized CD123×CD3 bi-specific monovalent diabody “DART-A,” that is capable of simultaneous binding to CD123 and CD3.

In detail, the invention provides a method of treating a hematologic malignancy comprising administering a CD123×CD3 binding molecule to a subject in need thereof, wherein:
(I) the CD123×CD3 binding molecule is a diabody consisting of a first polypeptide chain having the amino acid sequence of SEQ ID NO:21 and a second polypeptide chain having the amino acid sequence of SEQ ID NO:23; and
(II) the method comprises a 4-week treatment cycle (Cycle 1), wherein:
   (A) during days 1-3 of the first week of Cycle 1, the CD123×CD3 binding molecule is administered to the subject at a dosage of 30 ng/kg/day by continuous intravenous infusion;
   (B) during days 4-7 of the first week of Cycle 1, the CD123×CD3 binding molecule is administered to the subject at a dosage of 100 ng/kg/day by continuous infusion; and
   (C) during every day of weeks 2-4 of Cycle 1, the CD123×CD3 binding molecule is administered to the subject by continuous intravenous infusion at a single Treatment Dosage selected from the group consisting of the dosages: 300, 500, 700, 900 and 1000 ng/kg/day.

The invention additionally provides a CD123×CD3 binding molecule for use in the treatment of a hematologic malignancy, wherein:
(I) the CD123×CD3 binding molecule is a diabody consisting of a first polypeptide chain having the amino acid sequence of SEQ ID NO:21 and a second polypeptide chain having the amino acid sequence of SEQ ID NO:23; and
(II) the use comprises a 4-week treatment cycle (Cycle 1), wherein:
   (A) during days 1-3 of the first week of Cycle 1, the CD123×CD3 binding molecule is administered to the subject at a dosage of 30 ng/kg/day by continuous intravenous infusion;
   (B) during days 4-7 of the first week of Cycle 1, the CD123×CD3 binding molecule is administered to the subject at a dosage of 100 ng/kg/day by continuous infusion; and
   (C) during every day of weeks 2-4 of Cycle 1, the CD123×CD3 binding molecule is administered to the subject by continuous intravenous infusion at a single Treatment Dosage selected from the group consisting of the dosages: 300, 500, 700, 900 and 1000 ng/kg/day.

The invention additionally provides a method of treating a hematologic malignancy comprising administering a CD123×CD3 binding molecule to a subject in need thereof, wherein:
(I) the CD123×CD3 binding molecule is a diabody consisting of a first polypeptide chain having the amino acid sequence of SEQ ID NO:21 and a second polypeptide chain having the amino acid sequence of SEQ ID NO:23; and
(II) the method comprises a 4-week treatment cycle (Cycle 1), wherein:
   (A) during days 1-3 of the first week of the Cycle 1, the CD123×CD3 binding molecule is administered to the subject at a dosage of 30 ng/kg/day by continuous intravenous infusion;
   (B) during days 4-7 of the first week of the Cycle 1, the CD123×CD3 binding molecule is administered to the subject at a dosage of 100 ng/kg/day by continuous infusion;
   (C) during days 1-4 of weeks 2-4 of the Cycle 1, the CD123×CD3 binding molecule is administered to the subject by continuous intravenous infusion at a single Treatment Dosage selected from the group consisting of the dosages: 300, 500, 700, 900 and 1000 ng/kg/day; and
   (D) during days 5-7 of weeks 2-4 of Cycle 1, the subject is not provided with the CD123×CD3 binding molecule.

The invention additionally provides a CD123×CD3 binding molecule for use in the treatment of a hematologic malignancy, wherein:
(I) the CD123×CD3 binding molecule is a diabody consisting of a first polypeptide chain having the amino acid sequence of SEQ ID NO:21 and a second polypeptide chain having the amino acid sequence of SEQ ID NO:23; and
(II) the method comprises a 4-week treatment cycle (Cycle 1), wherein:
   (A) during days 1-3 of the first week of the Cycle 1, the CD123×CD3 binding molecule is administered to the subject at a dosage of 30 ng/kg/day by continuous intravenous infusion;
   (B) during days 4-7 of the first week of the Cycle 1, the CD123×CD3 binding molecule is administered to the subject at a dosage of 100 ng/kg/day by continuous infusion;
   (C) during days 1-4 of weeks 2-4 of the Cycle 1, the CD123×CD3 binding molecule is administered to the subject by continuous intravenous infusion at a single Treatment Dosage selected from the group consisting of the dosages: 300, 500, 700, 900 and 1000 ng/kg/day; and
   (D) during days 5-7 of weeks 2-4 of Cycle 1, the subject is not provided with the CD123×CD3 binding molecule.

The invention is additionally directed to the embodiment of such method and use wherein the Treatment Dosage is 300 ng/kg/day, or wherein the Treatment Dosage is 500 ng/kg/day, or wherein the Treatment Dosage is 700 ng/kg/day.

The invention is additionally directed to the embodiment of all of such above-indicated methods and uses wherein the treatment cycle (Cycle 1) is followed by one or more than one additional 4-week treatment cycle.

The invention is additionally directed to the embodiment of all such above-indicated methods and uses, wherein during days 1-4 of each week of the one or more additional treatment cycles, the selected Treatment Dosage of said CD123×CD3 binding molecule is administered to the subject by continuous intravenous infusion and during days 5-7 of each week of the one or more additional treatment cycles, the subject is not provided with the CD123×CD3 binding molecule.

The invention is additionally directed to the embodiment of all of such above-indicated methods and uses wherein dexamethasone is administered prophylactically.

The invention is additionally directed to the embodiment of all of such above-indicated methods and uses wherein the hematologic malignancy is selected from the group consisting of: acute myeloid leukemia (AML), chronic myelogenous leukemia (CML), including blastic crisis of CML and Abelson oncogene associated with CML (Bcr-ABL translocation), myelodysplastic syndrome (MDS), acute B lymphoblastic leukemia (B-ALL), chronic lymphocytic leukemia (CLL), including Richter's syndrome or Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.

The invention is additionally directed to the embodiment of all of such above-indicated methods and uses wherein the hematologic malignancy is acute myeloid leukemia.

The invention is additionally directed to the embodiment of all of such above-indicated methods and uses wherein the hematologic neoplasm is myelodysplastic syndrome.

The invention is additionally directed to the embodiment of all of such above-indicated methods and uses wherein the subject is a human.

### BRIEF DESCRIPTION OF THE DRAWINGS:

FIG. 1 shows that CD123 was known to be expressed on leukemic stem cells.

FIG. 2 illustrates the overall structure of the first and second polypeptide chains of two chain CD123×CD3 bi-specific monovalent diabodies, such as DART-A.

FIGS. 3A-3D show the activity of the CD123×CD3 DART® molecules of the present invention on PMBCs of AML patients. Primary PBMCs (containing 82% blasts) were treated with DART-A, a FITC×CD3 control DART® molecule, or phosphate buffered saline (PBS) for 144 hours. The E:T cell ratio was approximately 1:300 as determined from blast and T cell percentages in PBMCs at the start of the study. FIG. 3A: absolute number of leukemic blast cells (CD45+/CD33+); FIG. 3B: absolute numbers of T cells (CD4+ and CD8+); FIG. 3C: T-cell activation; FIG. 3D: cytokines measured in culture supernatants.

FIGS. 4A-4D show an overview of the CRS grade exhibited by participants in a Multi-Patient Dose Escalation Segment and Dose Expansion Phase Study, Cohort 3 ng (no prophylaxis) and Cohort 10 ng (no prophylaxis) (FIG. 4A); Cohort 30 ng (prophylactic dexamethasone), Cohort 100 ng (prophylactic dexamethasone), and Cohort 100/300 ng (prophylactic dexamethasone) (FIG. 4B); Cohort 100/500 ng (prophylactic dexamethasone+anti-cytokine) (FIG. 4C); Cohort LID/300 ng (prophylactic dexamethasone+anti-cytokine+2-step LID, continuous dosing schedule) and Cohort LID/500 ng (prophylactic dexamethasone+anti-cytokine+2-step LID, intermittent dosing schedule) (FIG. 4D).

FIG. 5 shows the anti-leukemic activity of 14 patients treated at the ≥500 ng/kg/day that received at least one cycle of treatment and had a post-treatment bone marrow biopsy (CR, Complete Response; CRm, molecular CR; CRi, Complete Response with incomplete hematological improvement; MLF=Morphologic Leukemia-free state; PR, Partial Response; SD/OB, Stable Disease/Other Anti-Leukemic Benefit; PD, Progressive Disease).

FIGS. 6A-6B show the ability of DART-A to mediate a reduction in AML blasts for three patients (Patient 1: AML MO; Refractory >2 Induction TX (open circles); Patient 2: AML M2; PR duration <6 months; Refractory to 2 salvage attempts (black circles) and Patient 3: AML FLT3 mutated; CR duration <6 months (gray circles)) in peripheral blood (FIG. 6A) and bone marrow (FIG. 6B).

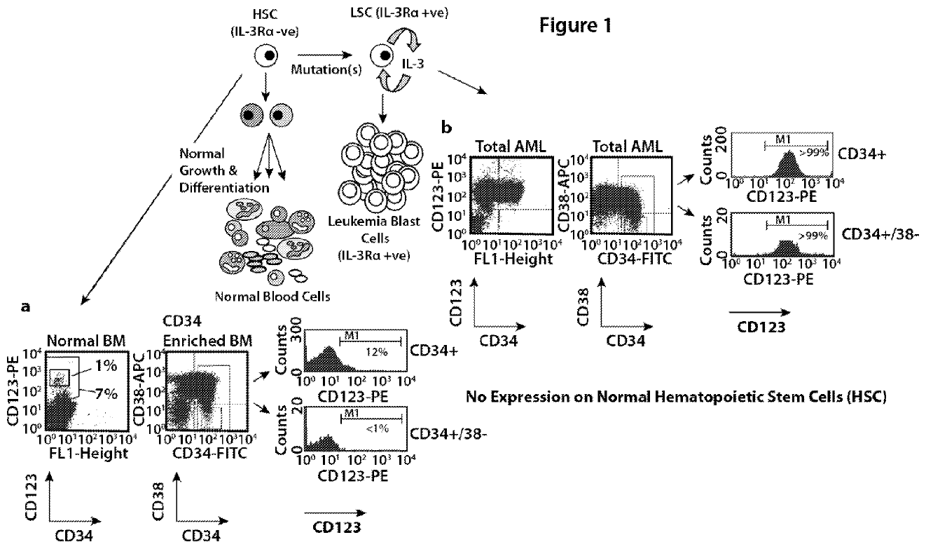

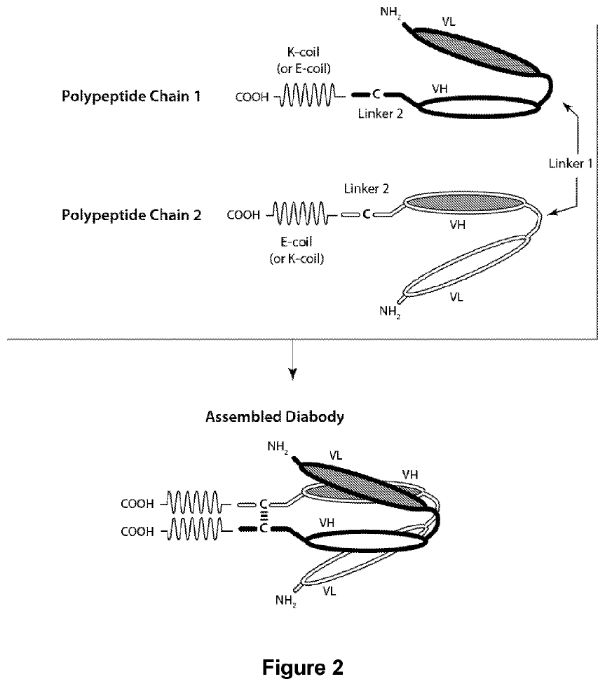

### DETAILED DESCRIPTION OF THE INVENTION:

The present invention is directed to a dosing regimen for administering a CD123×CD3 bi-specific monovalent diabody to patients with a hematologic malignancy such as acute myeloid leukemia (AML) or myelodysplastic syndrome (MDS). The invention particularly concerns the use of such a regimen for the sequence-optimized CD123×CD3 bi-specific monovalent diabody “DART-A,” that is capable of simultaneous binding to CD123 and CD3.

I. The Polypeptide Chains of DART-A

DART-A is a sequence-optimized bi-specific diabody capable of simultaneously and specifically binding to an epitope of CD123 and to an epitope of CD3 (a “CD123×CD3” bi-specific diabody) (US Patent Publn. No. US 2016-0200827, in PCT Publn. WO 2015/026892, in Al-Hussaini, M. et al. (2016) “Targeting CD123 In Acute Myeloid Leukemia Using A T-Cell-Directed Dual-Affinity Retargeting Platform,” Blood 127:122-131, in Vey, N. et al. (2017) “A Phase 1, First-in-Human Study of MGD006/S80880 (CD123×CD3) in AML/MDS,” 2017 ASCO Annual Meeting, Jun. 2-6, 2017, Chicago, Ill: Abstract TPS7070, each of which documents is herein incorporated by reference in its entirety). DART-A was found to exhibit enhanced functional activity relative to other non-sequence-optimized CD123×CD3 bi-specific diabodies of similar composition, and is thus termed a “sequence-optimized” CD123×CD3 bi-specific diabody.

DART-A comprises a first polypeptide chain and a second polypeptide chain. The first polypeptide chain of the bi-specific diabody will comprise, in the N-terminal to C-terminal direction, an N-terminus, a Light Chain Variable Domain (VL Domain) of a monoclonal antibody capable of binding to CD3 (VL_{CD3}), an intervening linker peptide (Linker 1), a Heavy Chain Variable Domain (VH Domain) of a monoclonal antibody capable of binding to CD123 (VH_{CD123}), and a C-terminus. A preferred sequence for such a VL_{CD3} Domain is SEQ ID NO:1:

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG

The Antigen Binding Domain of VL_{CD3} comprises CDR1 SEQ ID NO:2: RSSTGAVTTSNYAN, CDR2 SEQ ID NO:3: GTNKRAP, and CDR3 SEQ ID NO:4: ALWYSNLWV.

A preferred sequence for such Linker 1 is SEQ ID NO:5: GGGSGGGG. A preferred sequence for such a VH_{CD123} Domain is SEQ ID NO:6:

EVQLVQSGAE LKKPGASVKV SCKASGYTFT DYYMKWVRQA
PGQGLEWIGD IIPSNGATFY NQKFKGRVTI TVDKSTSTAY
MELSSLRSED TAVYYCARSH LLRASWFAYW GQGTLVTVSS

The Antigen Binding Domain of VH_{CD123} comprises CDR1 SEQ ID NO:7: DYYMK, CDR2 SEQ ID NO:8: DI IPSNGAT FYNQKFKG, and CDR3 SEQ ID NO:9: SHLLRAS.

The second polypeptide chain will comprise, in the N-terminal to C-terminal direction, an N-terminus, a VL domain of a monoclonal antibody capable of binding to CD123 (VLcm23), an intervening linker peptide (e.g., Linker 1), a VH domain of a monoclonal antibody capable of binding to CD3 (VHcD3), and a C-terminus. A preferred sequence for such a VL_{CD123} Domain is SEQ ID NO:10:

DFVMTQSPDS LAVSLGERVT MSCKSSQSLL NSGNQKNYLT
WYQQKPGQPP KLLIYWASTR ESGVPDRFSG SGSGTDFTLT
ISSLQAEDVA VYYCQNDYSY PYTFGQGTKL EIK

The Antigen Binding Domain of VL_{CD123} comprises CDR1 SEQ ID NO:11: KSSQSLLNSGNQKNYLT, CDR2 SEQ ID NO:12: WASTRELS, and CDR3 SEQ ID NO:13: QNDYSYPYT.

A preferred sequence for such a VH_{CD3} Domain is SEQ ID NO:14:

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL
VTVSS

The Antigen Binding Domain of VHCD3 comprises CDR1 SEQ ID NO:15: TAMM, CDR2 SEQ ID NO:16: RIRSKYNNYATYYADSVKD, and CDR3 SEQ ID NO:17: HGNFGNSYVSWFAY.

The sequence-optimized CD123×CD3 bi-specific diabodies of the present invention are engineered so that such first and second polypeptides covalently bond to one another via cysteine residues along their length. Such cysteine residues may be introduced into the intervening linker (e.g., Linker 1) that separates the VL and VH domains of the polypeptides. Alternatively, and more preferably, a second peptide (Linker 2) is introduced into each polypeptide chain, for example, at a position N-terminal to the VL domain or C-terminal to the VH domain of such polypeptide chain. A preferred sequence for such Linker 2 is SEQ ID NO:18: GGCGGG.

The formation of heterodimers can be driven by further engineering such polypeptide chains to contain polypeptide coils of opposing charge. Thus, in a preferred embodiment, one of the polypeptide chains will be engineered to contain an “E-coil” domain (SEQ ID NO:19: EVAALEKEVAALEKEVAALEKEVAALEK) whose residues will form a negative charge at pH 7, while the other of the two polypeptide chains will be engineered to contain an “K-coil” domain (SEQ ID NO:20: KVAALKEKVAALKEKVAALKEKVAALKE) whose residues will form a positive charge at pH 7. The presence of such charged domains promotes association between the first and second polypeptides, and thus fosters heterodimerization.

It is immaterial which coil is provided to the first or second polypeptide chains. However, a preferred sequence-optimized CD123×CD3 bi-specific diabody of the present invention (“DART-A”) has a first polypeptide chain having the sequence (SEQ ID NO:21):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
QLVQSGAELK KPGASVKVSC KASGYTFTDY YMKWVRQAPG
QGLEWIGDII PSNGATFYNQ KFKGRVTITV DKSTSTAYME
LSSLRSEDTA VYYCARSHLL RASWFAYWGQ GTLVTVSSGG
CGGGEVAALE KEVAALEKEV AALEKEVAAL EK

DART-A Chain 1 is composed of: SEQ ID NO:1-SEQ ID NO:5-SEQ ID NO:6-SEQ ID NO:18-SEQ ID NO:19. A DART-A Chain 1 encoding polynucleotide is SEQ ID NO:22:

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggcgg aggcgaggtg
cagctggtgc agtccggggc tgagctgaag aaacccggag
cttccgtgaa ggtgtcttgc aaagccagtg gctacacctt
cacagactac tatatgaagt gggtcaggca ggctccagga
cagggactgg aatggatcgg cgatatcatt ccttccaacg
gggccacttt ctacaatcag aagtttaaag gcagggtgac
tattaccgtg gacaaatcaa caagcactgc ttatatggag
ctgagctccc tgcgctctga agatacagcc gtgtactatt
gtgctcggtc acacctgctg agagccagct ggtttgctta
ttggggacag ggcaccctgg tgacagtgtc ttccggagga
tgtggcggtg gagaagtggc cgcactggag aaagaggttg
ctgctttgga gaaggaggtc gctgcacttg aaaaggaggt
cgcagccctg gagaaa

The second polypeptide chain of DART-A has the sequence (SEQ ID NO:23)

DFVMTQSPDS LAVSLGERVT MSCKSSQSLL NSGNQKNYLT
WYQQKPGQPP KLLIYWASTR ESGVPDRFSG SGSGTDFTLT
ISSLQAEDVA VYYCQNDYSY PYTFGQGTKL EIKGGGSGGG
GEVQLVESGG GLVQPGGSLR LSCAASGFTF STYAMNWVRQ
APGKGLEWVG RIRSKYNNYA TYYADSVKDR FTISRDDSKN
SLYLQMNSLK TEDTAVYYCV RHGNFGNSYV SWFAYWGQGT
LVTVSSGGCG GGKVAALKEK VAALKEKVAA LKEKVAALKE

DART-A Chain 2 is composed of: SEQ ID NO:10-SEQ ID NO:5-SEQ ID NO:14-SEQ ID NO:18-SEQ ID NO:20. A DART-A Chain 2 encoding polynucleotide is SEQ ID NO:24:

gacttcgtga tgacacagtc tcctgatagt ctggccgtga
gtctggggga gcgggtgact atgtcttgca agagctccca
gtcactgctg aacagcggaa atcagaaaaa ctatctgacc
tggtaccagc agaagccagg ccagccccct aaactgctga
tctattgggc ttccaccagg gaatctggcg tgcccgacag
attcagcggc agcggcagcg gcacagattt taccctgaca
atttctagtc tgcaggccga ggacgtggct gtgtactatt
gtcagaatga ttacagctat ccctacactt tcggccaggg
gaccaagctg gaaattaaag gaggcggatc cggcggcgga
ggcgaggtgc agctggtgga gtctggggga ggcttggtcc
agcctggagg gtccctgaga ctctcctgtg cagcctctgg
attcaccttc agcacatacg ctatgaattg ggtccgccag
gctccaggga aggggctgga gtgggttgga aggatcaggt
ccaagtacaa caattatgca acctactatg ccgactctgt
gaaggataga ttcaccatct caagagatga ttcaaagaac
tcactgtatc tgcaaatgaa cagcctgaaa accgaggaca
cggccgtgta ttactgtgtg agacacggta acttcggcaa
ttcttacgtg tcttggtttg cttattgggg acaggggaca
ctggtgactg tgtcttccgg aggatgtggc ggtggaaaag
tggccgcact gaaggagaaa gttgctgctt tgaaagagaa
ggtcgccgca cttaaggaaa aggtcgcagc cctgaaagag

II. The Properties of DART-A

DART-A was found to have the ability to simultaneously bind CD123 and CD3 as arrayed by human and cynomolgus monkey cells. Provision of DART-A was found to cause T cell activation, to mediate blast reduction, to drive T cell expansion, to induce T cell activation and to cause the redirected killing of target cancer cells (Table 1).

TABLE 1
Equilibrium Dissociation Constants (K_{D}) for the Binding
of DART-A to Human and Cynomolgus Monkey CD3 and CD123

| | kₐ (±SD) | k_{d} (±SD) | K_{D} (±SD) |
|---|---|---|---|
| Antigens | (M⁻¹s⁻¹) | (s⁻¹) | (nM) |
| Human CD3ε/δ | 5.7 (±0.6) × 10⁵ | 5.0 (±0.9) × 10⁻³ | 9.0 ± 2.3 |
| Cynomolgus CD3ε/δ | 5.5 (±0.5) × 10⁵ | 5.0 (±0.9) × 10⁻³ | 9.2 ± 2.3 |
| Human CD123-His | 1.6 (±0.4) × 10⁵ | 1.9 (±0.4) × 10⁻⁴ | 0.13 ± 0.01 |
| Cynomolgus CD123-His | 1.5 (±0.3) × 10⁵ | 4.0 (±0.7) × 10⁻⁴ | 0.27 ± 0.02 |

More particularly, DART-A was found to exhibit a potent redirected killing ability with concentrations required to achieve 50% of maximal activity (EC50s) in sub-ng/mL range, regardless of CD3 epitope binding specificity in target cell lines with high CD123 expression (Kasumi-3 (EC50=0.01 ng/mL)) medium CD123-expression (Molm13 (EC50=0.18 ng/mL) and THP-1 (EC50=0.24 ng/mL)) and medium low or low CD123 expression (TF-1 (EC50=0.46 ng/mL) and RS4-11 (EC50=0.5 ng/mL)). Similarly, DART-A-redirected killing was also observed with multiple target cell lines with T cells from different donors and no redirected killing activity was observed in cell lines that do not express CD123. Results are summarized in Table 2.

TABLE 2

| | | EC50 of Sequence- | |
|---|---|---|---|
| | CD123 surface | optimized CD123 × | |
| | expression | CD3 bi-specific | |
| | (antibody binding | diabodies (ng/mL) | |
| Target cell line | sites) | E:T = 10:1 | Max % killing |
| Kasumi-3 | 118620 | 0.01 | 94 |
| Molm13 | 27311 | 0.18 | 43 |
| THP-1 | 58316 | 0.24 | 40 |
| TF-1 | 14163 | 0.46 | 46 |
| RS4-11 | 957 | 0.5 | 60 |
| A498 | Negative | No activity | No activity |
| HT29 | Negative | No activity | No activity |

Additionally, when human T cells and tumor cells (Molm13 or RS4-11) were combined and injected subcutaneously into NOD/SCID gamma (NSG) knockout mice, the MOLM13 tumors was significantly inhibited at the 0.16, 0.5, 0.2, 0.1, 0.02, and 0.004 mg/kg dose levels. A dose of 0.004 mg/kg and higher was active in the MOLM13 model. The lower DART-A doses associated with the inhibition of tumor growth in the MOLM13 model compared with the RS4-11 model are consistent with the in vitro data demonstrating that MOLM13 cells have a higher level of CD123 expression than RS4-11 cells, which correlated with increased sensitivity to DART-A mediated cytotoxicity in vitro in MOLM13 cells.

DART-A was found to be active against primary AML specimens (bone marrow mononucleocytes (BMNC) and peripheral blood mononucleocytes (PBMC)) from AML patients. Incubation of primary AML bone marrow samples with DART-A resulted in depletion of the leukemic cell population over time, accompanied by a concomitant expansion of the residual T cells (both CD4 and CD8) and the induction of T cell activation markers (CD25 and Ki-67). Upregulation of granzyme B and perforin levels in both CD8 and CD4 T cells was observed. Incubation of primary ALL bone marrow samples with DART-A resulted in depletion of the leukemic cell population over time compared to untreated control or Control DART. When the T cells were counted (CD8 and CD4 staining) and activation (CD25 staining) were assayed, the T cells expanded and were activated in the DART-A sample compared to untreated or Control DART samples. DART-A was also found to be capable of mediating the depletion of pDCs cells in both human and cynomolgus monkey PBMCs, with cynomolgus monkey pDCs being depleted as early as 4 days post infusion with as little as 10 ng/kg DART-A. No elevation in the levels of cytokines interferon-gamma, TNF-alpha, IL6, ILS, IL4 and IL2 were observed in DART-A-treated animals. These data indicate that DART-A-mediated target cell killing was mediated through a granzyme B and perforin pathway.

No activity was observed against CD123-negative targets (U937 cells) or with Control DART, indicating that the observed T cell activation was strictly dependent upon target cell engagement and that monovalent engagement of CD3 by DART-A was insufficient to trigger T cell activation.

In sum, DART-A is an antibody-based molecule engaging the CD3E subunit of the TCR to redirect T lymphocytes against cells expressing CD123, an antigen up-regulated in several hematologic malignancies. DART-A binds to both human and cynomolgus monkey's antigens with similar affinities and redirects T cells from both species to kill CD123+ cells. Monkeys infused 4 or 7 days a week with weekly escalating doses of DART-A showed depletion of circulating CD123+ cells 72 h after treatment initiation that persisted throughout the 4 weeks of treatment, irrespective of dosing schedules. A decrease in circulating T cells also occurred, but recovered to baseline before the subsequent infusion in monkeys on the 4-day dose schedule, consistent with DART-A-mediated mobilization. DART-A administration increased circulating PD1+ but not TIM-3+ T cells; furthermore, ex vivo analysis of T cells from treated monkeys exhibited unaltered redirected target cell lysis, indicating no exhaustion. Toxicity was limited to a minimal transient release of cytokines following the DART-A first infusion, but not after subsequent administrations even when the dose was escalated, and a minimal reversible decrease in red cell mass with concomitant reduction in CD123+ bone marrow progenitors.

III. Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of DART-A and a pharmaceutically acceptable carrier.

The invention also encompasses pharmaceutical compositions comprising DART-A and a second therapeutic antibody (e.g., tumor specific monoclonal antibody) that is specific for a particular cancer antigen, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The invention also provides a pharmaceutical pack or kit comprising one or more containers containing DART-A alone or with such pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The present invention provides kits that comprise DART-A and that can be used in the above methods. In such kits, the DART-A is preferably packaged in a hermetically sealed container such as an ampoule or sachette indicating the quantity of the molecule. In one embodiment, the DART-A of such kit is supplied as a dry sterilized lyophilized powder or water-free concentrate in a hermetically sealed container and can be reconstituted, e.g., with water or saline to the appropriate concentration for administration to a subject. The lyophilized material should be stored at between 2 and 8° C. in their original container and the material should be administered within 12 hours, preferably within 6 hours, within 5 hours, within 3 hours, or within 1 hour after being reconstituted. The kit can further comprise one or more other prophylactic and/or therapeutic agents useful for the treatment of cancer, in one or more containers; and/or the kit can further comprise one or more cytotoxic antibodies that bind one or more cancer antigens associated with cancer. In certain embodiments, the other prophylactic or therapeutic agent is a chemotherapeutic. In other embodiments, the prophylactic or therapeutic agent is a biological or hormonal therapeutic.

IV. Methods of Administration

The compositions of the present invention may be provided for the treatment, prophylaxis, and amelioration of one or more symptoms associated with a disease, disorder or infection by administering to a subject an effective amount of a fusion protein or a conjugated molecule of the invention, or a pharmaceutical composition comprising a fusion protein or a conjugated molecule of the invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Methods of administering a molecule of the invention include, but are not limited to, parenteral administration (e.g., intradermal, intramuscular, intraperitoneal, intravenous and subcutaneous). In a specific embodiment, the sequence-optimized CD123×CD3 bi-specific diabodies of the invention are administered intravenously. The compositions may be administered by any convenient route, for example, by infusion or bolus injection, and may be administered together with other biologically active agents.

The amount of the composition of the invention which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems. Such dosages are preferably determined based upon the body weight (kg) of the recipient subject.

Preferably, DART-A will be administered to recipient human patients in one or more 4-week “cycles” in which a “lead-in” dosing strategy is used for the first week of the first cycle. Thus, in the initial week of the first cycle the recipient human patients will be administered a “lead-in dose” (“LID”) of 30 ng/kg/day of DART-A for 3 days, followed by 100 ng/kg/day of DART-A for 4 days.

In one embodiment, patients having received the LID (30 ng/kg/day ofDART-A for 3 days, followed by 100 ng/kg/day of DART-A for 4 days) are provided with additional DART-A using an Intermittent Dosing Schedule. In this embodiment, DART-A (at a dosage of either 500, 700, 900 or 1000 ng/kg/day) is provided to the patients by continuous infusion for 4 days, followed by 3 days without provision of DART-A, for weeks 2-4 of Cycle 1 and beyond (Table 3). Treatment at the same dosage and route of administration is continued until attainment of: (a) a complete response, (b) 1-2 cycles after attainment of complete response, (c) for a maximum of 12 cycles, (d) DLT, or (e) treatment failure. Clinical Responses (CRS) is preferably graded according to Lee criteria. Response is preferably assessed by International Working Group IWG (AML) or IPSS (MDS) criteria.

TABLE 3

| | Cycle | Intermittent Dosing |
|---|---|---|
| Cycle | Week | Schedule |
| 1 | 1 | 7 Days Lead-In |
| | 2 | 4 days on/3 days off |
| | 3 | 4 days on/3 days off |
| | 4 | 4 days on/3 days off |
| 2 and >2 | 1 | 4 days on/3 days off |
| | 2 | 4 days on/3 days off |
| | 3 | 4 days on/3 days off |
| | 4 | 4 days on/3 days off |

In an alternative embodiment, patients having received the LID (30 ng/kg/day of DART-A for 3 days, followed by 100 ng/kg/day ofDART-A for 4 days) are provided with additional DART-A using a Continuous Dosing Schedule. In this embodiment, DART-A (at a dosage of either 300, 500, 700, 900 or 1000 ng/kg/day) is provided to the patients by continuous infusion for 21 days (weeks 2-4), thereby completing the first cycle. Treatment is continued in subsequent cycles by alternating 4-day periods of continuous infusion administration of the same dosage of DART-A with 3-day periods without provision of DART-A (Table 4).

TABLE 4

| | Cycle | Continuous Dosing |
|---|---|---|
| Cycle | Week | Schedule |
| 1 | 1 | 7 Days Lead-In |
| | 2-4 | Continuous Dosing |
| 2 and >2 | 1 | 4 days on/3 days off |
| | 2 | 4 days on/3 days off |
| | 3 | 4 days on/3 days off |
| | 4 | 4 days on/3 days off |

In both Intermittent Dosing Schedule and the Continuous Dosing Schedule, treatment is continued until attainment of complete response, 1-2 cycles after attainment of complete response, for a maximum of 12 cycles, DLT, or treatment failure. CRS is preferably graded according to Lee criteria. Response (complete remission (CR), incomplete blood count recovery (Cri), partial remission(PR) or improvement in peripheral blood and bone marrow (PB/BM) AML blast count) is preferably assessed by International Working Group IWG (AML) or IPS S (MDS) criteria.

V. Uses of the Compositions of the Invention

DART-A may be used to treat any disease or condition associated with or characterized by the expression of CD123. In particular, DART-A may be used to treat hematologic malignancies. Thus, without limitation, such molecules may be employed in the diagnosis or treatment of acute myeloid leukemia (AML), chronic myelogenous leukemia (CML), including blastic crisis of CML and Abelson oncogene associated with CML (Bcr-ABL translocation), myelodysplastic syndrome (MDS), acute B lymphoblastic leukemia (B-ALL), chronic lymphocytic leukemia (CELL), including Richter's syndrome or Richter's transformation of call, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma. DART-A may additionally be used in the manufacture of medicaments for the treatment of the above-described conditions.

VI. Embodiments of the Invention

The invention is directed to the following embodiments (E):
E(1). A method of treating a hematologic malignancy comprising administering a CD123×CD3 binding molecule to a subject in need thereof, wherein:
   (I) the CD123×CD3 binding molecule is a diabody consisting of a first polypeptide chain having the amino acid sequence of SEQ ID NO:21 and a second polypeptide chain having the amino acid sequence of SEQ ID NO:23; and
   (II) the method comprises a 4-week treatment cycle (Cycle 1), wherein:
      (A) during days 1-3 of the first week of the Cycle 1, the CD123×CD3 binding molecule is administered to the subject at a dosage of 30 ng/kg/day by continuous intravenous infusion;
      (B) during days 4-7 of the first week of the Cycle 1, the CD123×CD3 binding molecule is administered to the subject at a dosage of 100 ng/kg/day by continuous infusion; and
      (C) during every day of weeks 2-4 of the Cycle 1, the CD123×CD3 binding molecule is administered to the subject by continuous intravenous infusion at a single Treatment Dosage selected from the group consisting of the dosages: 300, 500, 700, 900 and 1000 ng/kg/day.
E(2) A CD123×CD3 binding molecule for use in the treatment of a hematologic malignancy, wherein:
   (I) the CD123×CD3 binding molecule is a diabody consisting of a first polypeptide chain having the amino acid sequence of SEQ ID NO:21 and a second polypeptide chain having the amino acid sequence of SEQ ID NO:23; and
   (II) the use comprises a 4-week treatment cycle (Cycle 1), wherein:
      (A) during days 1-3 of the first week of the Cycle 1, the CD123×CD3 binding molecule is administered to the subject at a dosage of 30 ng/kg/day by continuous intravenous infusion;
      (B) during days 4-7 of the first week of the Cycle 1, the CD123×CD3 binding molecule is administered to the subject at a dosage of 100 ng/kg/day by continuous infusion; and
      (C) during every day of weeks 2-4 of the Cycle 1, the CD123×CD3 binding molecule is administered to the subject by continuous intravenous infusion at a single Treatment Dosage selected from the group consisting of the dosages: 300, 500, 700, 900 and 1000 ng/kg/day.
E(3) A method of treating a hematologic malignancy comprising administering a CD123×CD3 binding molecule to a subject in need thereof, wherein:
   (I) the CD123×CD3 binding molecule is a diabody consisting of a first polypeptide chain having the amino acid sequence of SEQ ID NO:21 and a second polypeptide chain having the amino acid sequence of SEQ ID NO:23; and
   (II) the method comprises a 4-week treatment cycle (Cycle 1), wherein:
      (A) during days 1-3 of the first week of the Cycle 1, the CD123×CD3 binding molecule is administered to the subject at a dosage of 30 ng/kg/day by continuous intravenous infusion;
      (B) during days 4-7 of the first week of the Cycle 1, the CD123×CD3 binding molecule is administered to the subject at a dosage of 100 ng/kg/day by continuous infusion;
      (C) during days 1-4 of weeks 2-4 of the Cycle 1, the CD123×CD3 binding molecule is administered to the subject by continuous intravenous infusion at a single Treatment Dosage selected from the group consisting of the dosages: 300, 500, 700, 900 and 1000 ng/kg/day;
      (D) during days 5-7 of weeks 2-4 of Cycle 1, the subject is not provided with the CD123×CD3 binding molecule.
E(4) A CD123×CD3 binding molecule for use in the treatment of a hematologic malignancy, wherein:
   (I) the CD123×CD3 binding molecule is a diabody consisting of a first polypeptide chain having the amino acid sequence of SEQ ID NO:21 and a second polypeptide chain having the amino acid sequence of SEQ ID NO:23; and
   (II) the method comprises a 4-week treatment cycle (Cycle 1), wherein:
      (A) during days 1-3 of the first week of the Cycle 1, the CD123×CD3 binding molecule is administered to the subject at a dosage of 30 ng/kg/day by continuous intravenous infusion;
      (B) during days 4-7 of the first week of the Cycle 1, the CD123×CD3 binding molecule is administered to the subject at a dosage of 100 ng/kg/day by continuous infusion;
      (C) during days 1-4 of weeks 2-4 of the Cycle 1, the CD123×CD3 binding molecule is administered to the subject by continuous intravenous infusion at a single Treatment Dosage selected from the group consisting of the dosages: 300, 500, 700, 900 and 1000 ng/kg/day;
      (D) during days 5-7 of weeks 2-4 of Cycle 1, the subject is not provided with the CD123×CD3 binding molecule.
E(5) The method of E(1) or E(3), or the use of E(2) or E(4), wherein the Treatment Dosage is 300 ng/kg/day.
E(6) The method of E(1) or E(3), or the use of E(2) or E(4), wherein the Treatment Dosage is 500 ng/kg/day.
E(7) The method of E(1) or E(3), or the use of E(2) or E(4), wherein the Treatment Dosage is 700 ng/kg/day.
E(8) The method of any of E(1), E(3) or E(5)-E(7), or the use of any of E(2)-E(7), wherein the treatment cycle (Cycle 1) is followed by one or more than one additional 4-week treatment cycle.
E(9) The method of E(8), or the use of E(8), wherein during days 1-4 of each week of said one or more additional treatment cycles, said selected Treatment Dosage of said CD123×CD3 binding molecule is administered to said subject by continuous intravenous infusion and during days 5-7 of each week of said one or more additional treatment cycles, said subject is not provided with said CD123×CD3 binding molecule.
E(10) The method of any of E(1), E(3) or E(5)-E(9), or the use of any of E(2) or E(4)-E(9), wherein dexamethasone is administered prophylactically.
E(11) The method of any of E(1), E(3) or E(5)-E(10), or the use of any of E(2) or E(4)-E(10), wherein the hematologic malignancy is selected from the group consisting of: acute myeloid leukemia (AML), chronic myelogenous leukemia (CML), including blastic crisis of CML and Abelson oncogene associated with CML (Bcr-ABL translocation), myelodysplastic syndrome (MDS), acute B lymphoblastic leukemia (B-ALL), chronic lymphocytic leukemia (CLL), including Richter's syndrome or Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.
E(12) The method of E(11), wherein the hematologic malignancy is acute myeloid leukemia.
E(13) The method of E(11), wherein the hematologic malignancy is myelodysplastic syndrome.
E(14) The method of any of E(1), E(3), E(5)-E(13), or the use of any of E(2) or E(4)-E(13), wherein the subject is a human.

### EXAMPLES

Having now generally described the invention, the same will be more readily understood through reference to the following examples, which are provided by way of illustration and are not intended to be limiting of the present invention unless specified.

### Example 1

### Activity of CD123×CD3 DART® Molecule in Primary AML Patient Samples

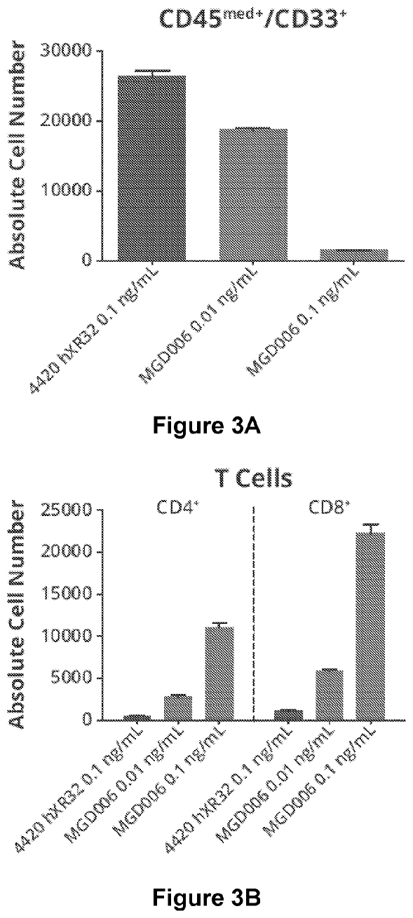

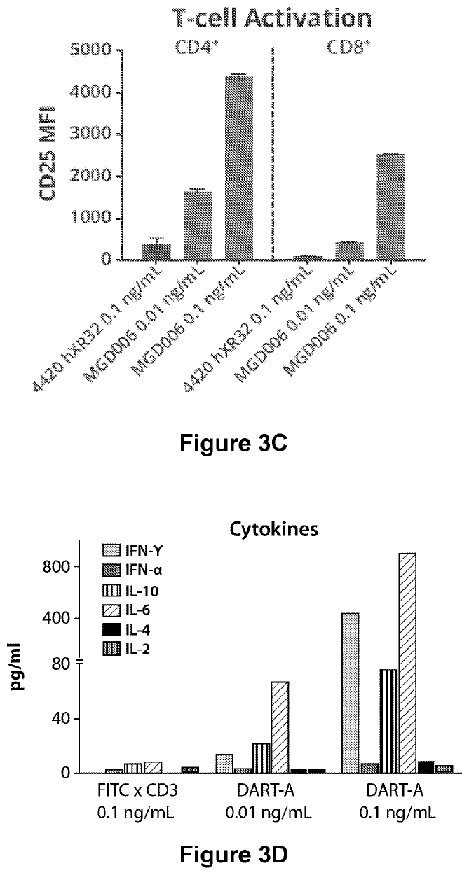

The ability of DART-A to kill CD123-expressing cells of primary AML patient samples was investigated. AML patient primary PBMCs (containing 82% blasts) were treated with a CD123×CD3 DART® molecule, a FITC×CD3 control DART® molecule, or phosphate buffered saline (PBS) for 144 hours. The E:T cell ratio was approximately 1:300 as determined from blast and T cell percentages in PBMCs at the start of the study. The absolute number of leukemic blast cells (CD45+ /CD33+) is shown in FIG. 3A. The absolute numbers of T cells (CD4+ and CD8+) are shown in FIG. 3B. FIG. 3C shows T-cell activation. Cytokines measured in culture supernatants are shown in FIG. 3D.

### Example 2

### Phase 1, First-in-Human Study of CD123×CD3 DART Diabody in AML and MDS

Acute myeloid leukemia (AML) is characterized by the expansion of CD34+, CD38⁻ cells with high levels of CD123, the alpha chain of the interleukin 3 receptor (IL-3Rα). CD123 is highly expressed in >90% of AML patients and at least 50% of MDS patients. CD123 expression in AML blasts has been related with high-risk disease and disease progression, enabling a promising strategy of preferential ablation with CD123 targeted approach. Because AML blast and leukemic stem cells highly express CD123, which is associated with high-risk disease and disease progression whereas CD123 expression on normal hematopoietic stem cells is minimal, AML (and myelodysplastic syndrome (MDS)) are reasonable targets for CD123-based immunotherapy.

The DART-A molecule of the present invention shows potent activity to target CD123-expressing cell lines and primary AML blasts in vitro for recognition and elimination by CD3-expressing T lymphocytes as effector cells, and are capable of inhibiting the growth of leukemic cell lines in mice and depleting CD123-positive plasmacytoid dendritic cells in cynomolgus macaques, and thus provide a strategy for the preferential ablation of AML with a CD123-targeted approach.

### Single-Patient Dose Escalation

In order to determine the tolerability of patients to DART-A, a Single-Patient Dose Escalation Study was conducted. Single patient mini-cohorts were dosed with a continuous IV infusion (CIV) using a lead-in dosing strategy of 3 ng/kg/day, followed by 10 ng/kg/day, followed by 30 ng/kg/day, followed by 100 ng/kg/day, with each such progression in dose occurring if dose-limiting toxicity (DLT) was less than 33%. The cohorts were increased to 4 patients if adverse effects (AE) ≥Grade 2. The results of this study indicated that DART-A was tolerated at all tested dosages.

### Lead-in Dose Optimization to Mitigate Cytokine Release Syndrome in AML and MDS Patients

Cytokine secretion with ensuing potential for cytokine release syndrome (CRS) is inherent in T-cell activation and a limiting toxicity with T-cell redirecting therapies. In the Phase 1 study of the ability of DART-A to mediate such T-cell activation in the treatment of AML and MDS, two lead-in dose (LID) strategies, in conjunction with early intervention with tocilizumab (Maude, S.L. et al. (2014) “Managing Cytokine Release Syndrome Associated with Novel T Cell-Engaging Therapies.” Cancer Journal 20:119-122), were compared for their ability to mitigate CRS.

In the first LID strategy (LID-1 schema), DART-A was administered at 100 ng/kg/day for 4 days followed by a 3 day pause and resumption of treatment at 300 or 500ng/kg/day starting on Day 8. In the second LID strategy (LID-2 schema), DART-A was administered at 30 ng/kg/day for 3 days, followed by 100 ng/kg/day for the next 4 days, followed by a dose of 300 ng/kg/day continuous dosing schedule or 500 ng/kg/day intermittent dosing schedule starting on Day 8.

In the evaluation of lead-in dose strategies, IL-2, IL-6, IL-8, IL-10, TNF-a, INF-γ, and GM-CSF were measured and CRS severity was graded. Peak cytokine values during first reported CRS events, occurring within 10 days of start of first dose, were evaluated. Median peak cytokine levels were compared between patients with and without LID. Other potential CRS determinants were evaluated.

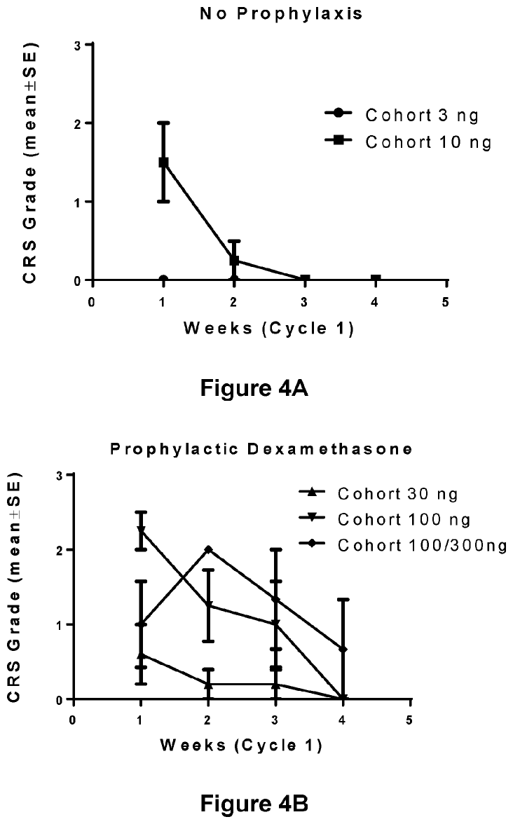

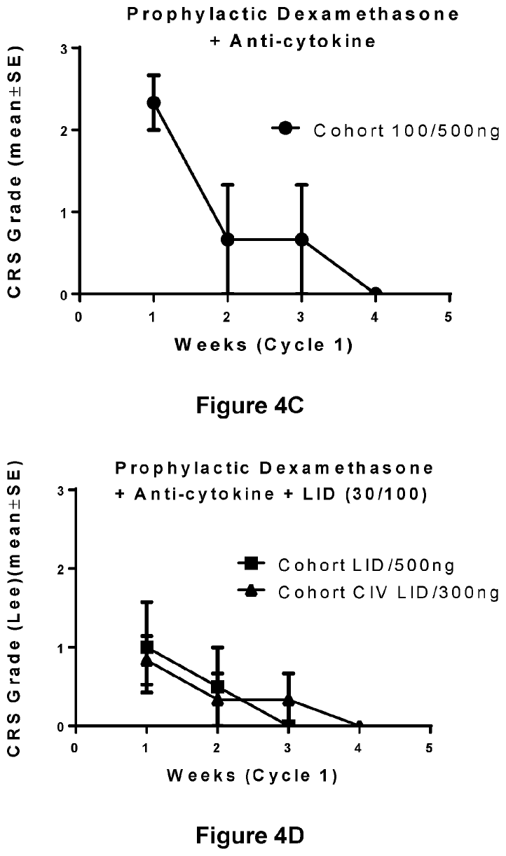

Infusion-related reaction (IRR)/CRS occurred in (76%) of patients, with most events (82%) ≤ Grade (Gr) 2, manageable and reversible. Among 29 patients with complete cytokine data, 68% experienced CRS within 2 days of start of DART-A, and an additional 8% within 10 days (14% Gr 1, 55% Gr2, and 7% Gr 3). Cytokine levels were generally higher in patients with CRS than in patients without CRS (median IL6, 116.2 vs. 67.9 pg/mL; IL8, 191.1, vs. 144.6 pg/mL; IL10, 867.6, vs. 348.7 pg/mL), and were generally higher with increasing CRS grade. The use of a LID reduced overall cytokine levels, with institution of the two-step LID in Week 1 decreasing severity by mean 0.54 grade during cycle 1 (mean CRS grade week 1, 1.16 vs. 2; week 2, 1 vs. 1.33; week 3, 0.67 vs. 0.83; week 4, 0.13 vs 0.67 LID-2 vs. LID-1, respectively). Median peak cytokine levels observed with the two-step LID were lower during Week 1 and after achieving maximum dose. Preliminary data show relation between baseline circulating T-cell number and maximum CRS grade during Week 1, with higher grade of CRS (≥2) in Week 1 associated with higher baseline levels of circulating T-cells. Other variables evaluated did not trend with CRS grade. CRS grade and frequency did not correlate with response. FIGS. 4A-4D present an overview of the CRS grade exhibited by study participants, and show that the introduction of the two step LID strategy (30 ng/kg/day for 3 days, followed by 100 ng/kg/day for the next 4 days) prior to administration of a step-up dose (treatment dose) of 300 or 500 ng/kg/day decreased CRS across the first study cycle.

In sum, CRS has been a limiting factor with T-cell directing therapies. The employed two-step LID showed effectiveness in limiting IRR/CRS events and circulating cytokines over a single-step LID.

To mitigate cytokine-release syndrome (CRS), a two-step LID (30 ng/kg/day for 3 days followed by 100 ng/kg/day for 4 days) was instituted during Cycle 1/Week 1 (C1W1), to be followed by the cohort target dose (300-1000 ng/kg/day) on either of the dosing schedules (continuous or intermittent) on W2-4. In Cycle 2 (C2) and beyond, all patients are treated on a 4-day on/3-day off schedule at the cohort target dose for a maximum of 12 cycles, with 2 cycles after a CR or CRi. Steroid-sparing, anti-cytokine (tocilizumab) therapy is used, if clinically indicated, to manage CRS symptoms. Disease status is assessed by IWG criteria. Samples are collected for PK, anti-drug antibodies (ADA) and cytokine analysis, including IL2, 11,6, IL8, IL10, TNF-alpha, IFN-gamma and GM-CSF. A post-treatment bone marrow biopsy may also be obtained.

In the Multi-Patient Dose Escalation Segment and Dose Expansion Phase Study were patients with relapsed/refractory AML and hypomethylation failure Int-2/High Risk MDS. Patients were dosed with a continuous IV infusion (CIV) in 28-day cycles. The above-described 2-step lead-in dosing strategy was employed (in which patients were administered DART-A at a dosage of 30 ng/kg/day for 3 days, followed by 100 ng/kg/day for 4 days), followed by a dose of 300 or 500 ng/kg/day on Day 8). In subsequent Weeks 2-4, two different dosage schedules were evaluated (an Intermittent Dosage Schedule (Table 3) and a Continuous Dosage Schedule (Table 4)), after which disease evaluation was conducted (complete remission (CR); molecular complete remission (CRm); incomplete blood count recovery (CRi); partial remission (PR); progressive disease (PD); or improvement in peripheral blood and bone marrow (PB/BM) AML blast count.

Beginning with the second cycle, all patients received the CD123×CD3 DART® molecule for 4 days on/3 days off at the maximal dose/cohort. The treatment was continued until 2 cycles after attainment of complete response, for a maximum of 12 cycles, DLT, or treatment failure, or for a maximum of 12 cycles. Cytokine Release Syndrome (CRS) was graded according to Lee criteria. Response was assessed by International Working Group IWG (AML) or IPSS (MDS) criteria.

Once an MTDS or MAD had been determined, dose expansion occurred with patients exhibiting relapsed/refractory R/R AML in one expansion cohort and patients with hypomethylation Failure MDS in a second expansion cohort. The enrolled additional patients were used to evaluate efficacy.

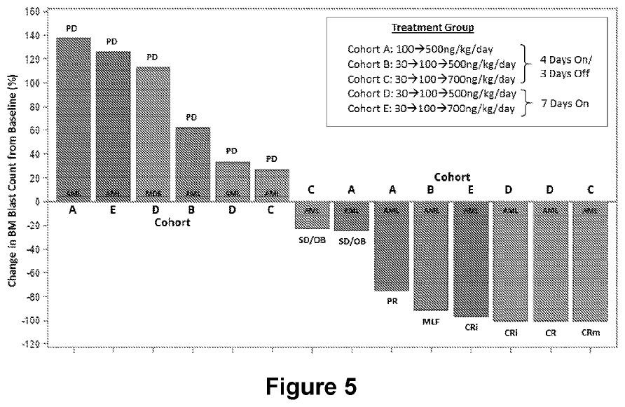

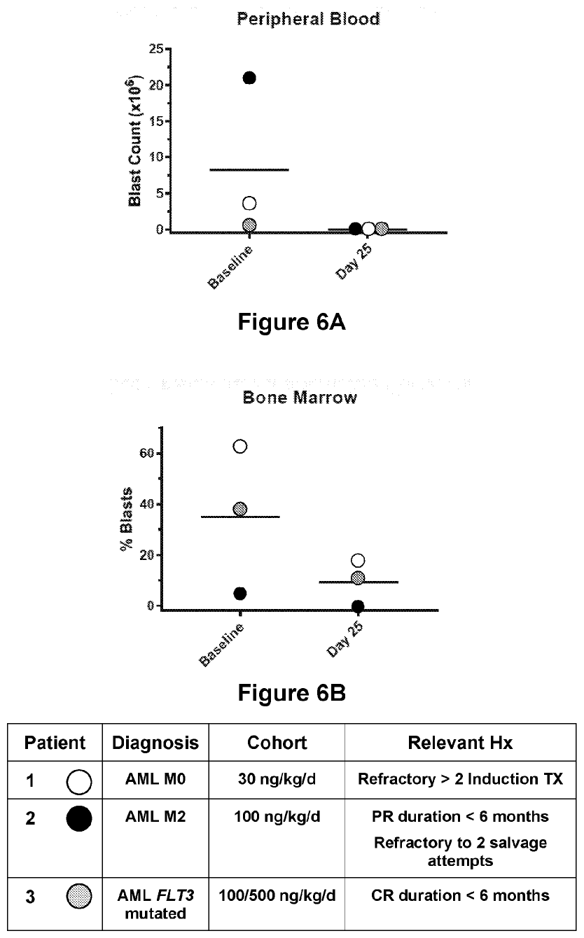

Forty-five (45) patients (median age of 64 (29-84), and 44% female) with R/R AML/MDS (89% AML and 11% MDS) were treated with DART-A. The MTDS was reached at 500 ng/kg/day. Overall, DART-A demonstrated manageable toxicity (drug-related adverse event ≥G3 were observed in 20/45 (44%) patients; infusion-related reaction/cytokine release syndrome (IRR/CRS) was the most common toxicity, and was observed in 34/45 (76%) patients (G3 in 6/45, 13%). The most frequent CRS symptoms were pyrexia (15), chills (10), tachycardia (10), and hypotension (4). Fourteen (14) patients treated at the threshold 500 ng/kg/day dose cohort and beyond (700 ng/kg/day dose cohort) completed at least one cycle of treatment and had a post-treatment bone marrow biopsy. Anti-leukemic activity was documented in 57% (8/14) patients, 6/14 reached IWG criteria (3 CR, 1 CRi, 1 MLF (morphologic leukemia free), 1 PR) for an objective response rate (ORR) of 43%, and 2 patients had stable disease and BM blast reduction of 20% and 25% from baseline (FIG. 5). Blast reduction occurred rapidly, often within one cycle of therapy and extended beyond DART-A discontinuation. FIGS. 6A-6B show the reduction in blast counts in three patients. Multispectral immunohistochemistry analysis of bone marrow samples showed DART-A in situ with a significant increase (in CD8 T cells (1.58-fold increase, p=0.0013). Consistent with T-cell activation, CD25, CD69 and PD-1 upregulation on both CD4 and CDS T-cells was also observed in peripheral blood samples.

In sum, a Phase 1 dose-escalation study was conducted in order to determine the maximum tolerated dose and schedule (MTDS) or maximum acceptable dose (MAD) and to describe the pharmacokinetics, safety and preliminary efficacy of the CD123×CD3 DART® molecules of the present invention. This Phase 1 study was designed to define the safety profile, maximum tolerated dose and schedule (MTDS), and preliminary anti-leukemic activity of DART-A. Relapsed/refractory (R/R) AML or intermediate-2/high-risk MDS patients were treated on 28-day cycles at doses from 3-1000 ng/kg/day on one of 2 dosing schedules (a Continuous Dosing Schedule or an Intermittent Dosing Schedule). DART-A was found to exhibit an acceptable safety profile and demonstrated evidence of anti-leukemic (ORR 43%) activity with manageable safety profile in the treatment of AML and MDS. Incorporation of a lead-in dosing strategy improved tolerability. DART-A may be used in treating/preventing antigen-loss relapse in ALL patients post CD19-CAR-T cells or blinatumomab.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of treating a hematologic malignancy comprising administering a CD123×CD3 binding molecule to a subject in need thereof, wherein:
(I) said CD123×CD3 binding molecule is a diabody consisting of a first polypeptide chain having the amino acid sequence of SEQ ID NO:21 and a second polypeptide chain having the amino acid sequence of SEQ ID NO:23; and
(II) said method comprises a 4-week treatment cycle (Cycle 1), wherein:
(A) during days 1-3 of the first week of said Cycle 1, said CD123×CD3 binding molecule is administered to said subject at a dosage of 30 ng/kg/day by continuous intravenous infusion;
(B) during days 4-7 of the first week of said Cycle 1, said CD123×CD3 binding molecule is administered to said subject at a dosage of 100 ng/kg/day by continuous infusion; and
(C) during every day of weeks 2-4 of said Cycle 1, said CD123×CD3 binding molecule is administered to said subject by continuous intravenous infusion at a single Treatment Dosage selected from the group consisting of the dosages: 300, 500, 700, 900 and 1000 ng/kg/day.

2. A CD123×CD3 binding molecule for use in the treatment of a hematologic malignancy, wherein:
(I) said CD123×CD3 binding molecule is a diabody consisting of a first polypeptide chain having the amino acid sequence of SEQ ID NO:21 and a second polypeptide chain having the amino acid sequence of SEQ ID NO:23; and
(II) said use comprises a 4-week treatment cycle (Cycle 1), wherein:
(A) during days 1-3 of the first week of said Cycle 1, said CD123×CD3 binding molecule is administered to said subject at a dosage of 30 ng/kg/day by continuous intravenous infusion;
(B) during days 4-7 of the first week of said Cycle 1, said CD123×CD3 binding molecule is administered to said subject at a dosage of 100 ng/kg/day by continuous infusion; and
(C) during every day of weeks 2-4 of said Cycle 1, said CD123×CD3 binding molecule is administered to said subject by continuous intravenous infusion at a single Treatment Dosage selected from the group consisting of the dosages: 300, 500, 700, 900 and 1000 ng/kg/day.

3. A method of treating a hematologic malignancy comprising administering a CD123×CD3 binding molecule to a subject in need thereof, wherein:
(I) said CD123×CD3 binding molecule is a diabody consisting of a first polypeptide chain having the amino acid sequence of SEQ ID NO:21 and a second polypeptide chain having the amino acid sequence of SEQ ID NO:23; and
(II) said method comprises a 4-week treatment cycle (Cycle 1), wherein:
(A) during days 1-3 of the first week of said Cycle 1, said CD123×CD3 binding molecule is administered to said subject at a dosage of 30 ng/kg/day by continuous intravenous infusion;
(B) during days 4-7 of the first week of said Cycle 1, said CD123×CD3 binding molecule is administered to said subject at a dosage of 100 ng/kg/day by continuous infusion;
(C) during days 1-4 of weeks 2-4 of said Cycle 1, said CD123×CD3 binding molecule is administered to said subject by continuous intravenous infusion at a single Treatment Dosage selected from the group consisting of the dosages: 300, 500, 700, 900 and 1000 ng/kg/day; and
(D) during days 5-7 of weeks 2-4 of Cycle 1, said subject is not provided with said CD123×CD3 binding molecule.

4. A CD123×CD3 binding molecule for use in the treatment of a hematologic malignancy, wherein:
(I) said CD123×CD3 binding molecule is a diabody consisting of a first polypeptide chain having the amino acid sequence of SEQ ID NO:21 and a second polypeptide chain having the amino acid sequence of SEQ ID NO:23; and
(II) said method comprises a 4-week treatment cycle (Cycle 1), wherein:
(A) during days 1-3 of the first week of said Cycle 1, said CD123×CD3 binding molecule is administered to said subject at a dosage of 30 ng/kg/day by continuous intravenous infusion;
(B) during days 4-7 of the first week of said Cycle 1, said CD123×CD3 binding molecule is administered to said subject at a dosage of 100 ng/kg/day by continuous infusion;
(C) during days 1-4 of weeks 2-4 of said Cycle 1, said CD123×CD3 binding molecule is administered to said subject by continuous intravenous infusion at a single Treatment Dosage selected from the group consisting of the dosages: 300, 500, 700, 900 and 1000 ng/kg/day; and
(D) during days 5-7 of weeks 2-4 of Cycle 1, said subject is not provided with said CD123×CD3 binding molecule.

5. The method of claim 1 or 3, or the use of claim 2 or 4, wherein said Treatment Dosage is 300 ng/kg/day.

6. The method of claim 1 or 3, or the use of claim 2 or 4, wherein said Treatment Dosage is 500 ng/kg/day.

7. The method of claim 1 or 3, or the use of claim 2 or 4, wherein said Treatment Dosage is 700 ng/kg/day.

8. The method of any of claim 1, 3 or 5-7, or the use of any of claim 2 or 4-7, wherein said treatment cycle (Cycle 1) is followed by one or more than one additional 4-week treatment cycle.

9. The method of claim 8, or the use of claim 8, wherein during days 1-4 of each week of said one or more additional treatment cycles, said selected Treatment Dosage of said CD123×CD3 binding molecule is administered to said subject by continuous intravenous infusion and during days 5-7 of each week of said one or more additional treatment cycles, said subject is not provided with said CD123×CD3 binding molecule.

10. The method of any of claim 1, 3 or 5-9, or the use of any of claim 2 or 4-9, wherein dexamethasone is administered prophylactically.

11. The method of any of claim 1,3 or 5-10, or the use of any of claim 2 or 4-10, wherein said hematologic malignancy is selected from the group consisting of: acute myeloid leukemia (AML), chronic myelogenous leukemia (CML), including blastic crisis of CML and Abelson oncogene associated with CML (Bcr-ABL translocation), myelodysplastic syndrome (MDS), acute B lymphoblastic leukemia (B-ALL), chronic lymphocytic leukemia (CLL), including Richter's syndrome or Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.

12. The method of claim 11, wherein said hematologic acute myeloid leukemia.

13. The method of claim 11, wherein said hematologic malignancy is myelodysplastic syndrome.

14. The method of any of claim 1, 3 or 5-13, or the use of any of claim 2, or 4-13, wherein said subject is a human.
